(19) United States
(12) Patent Application Publication
(10) Pub. No.: US 2018/0147381 A1
(43) Pub. Date: May 31, 2018

(54) Endotracheal Intubation System

(71) Applicant: Tien-Sheng Chen, Taipei City (TW)

(72) Inventor: Tien-Sheng Chen, Taipei City (TW)

(21) Appl. No.: 15/702,913

(22) Filed: Sep. 13, 2017

(30) Foreign Application Priority Data
Nov. 30, 2016 (TW) 105139542

(51) Int. Cl. A61M 16/04; A61B 1/267; A61B 1/05; A61B 1/00

(52) U.S. Cl. CPC A61M 16/0488; A61B 1/267; A61B 1/0005; A61B 1/00052; A61B 1/051

(57) ABSTRACT

An endotracheal tube has a tip. An endotracheal intubation system has a first camera, a blade, a second camera, and a display. The blade has a camera accommodation, a passage, and a guiding portion. The first camera is situated in the camera accommodation for capturing a first image. The passage has a side surface and a top surface. The guiding portion is the junction of the side surface and the top surface. The second camera is for capturing an image of the guiding portion when the endotracheal tube moves along the passage and is situated within the endotracheal tube. The display is electrically connected with the first camera and the second camera for displaying the first image and the image of the guiding portion to ensure that the tip of the endotracheal tube moves along the guiding portion when the endotracheal tube moves along the passage.

### BACKGROUND OF THE INVENTION

### 1. Field of the Invention

The present invention relates to an endotracheal intubation system; more particularly, the present invention relates to a device for positioning the tip of an endotracheal tube moving along a guiding portion continuously by using an image of the guiding portion captured by a camera when the endotracheal tube is moving, so as to accelerate the efficiency and success rate of intubation and to avoid injury to the patient.

### 2. Description of the Related Art

For patients with respiratory termination, tracheal intubation is a necessary means for maintaining respiratory function. In order to maintain the patient's life, a medical worker such as a doctor or an emergency medical technician must insert an endotracheal tube into the patient's trachea to provide oxygen in a short time. However, because the upper respiratory tract structures of patients vary due to different ages or body types, sometimes the medical worker cannot easily or directly observe a patient's tracheal position. Therefore, on most of the laryngoscopes currently in use, a camera is installed on a side of an endotracheal tube channel. After the medical worker observes the patient's endotracheal opening through the camera, the endotracheal tube is inserted through the endotracheal tube passage disposed at the side of the camera and into the patient's trachea.

However, there are still inconveniences to be overcome in using the laryngoscope of the prior art. Sometimes, because of differences in the body structures of patients, the endotracheal tube passage is obstructed by hypertrophic soft tissue or swollen tonsils of the patient; thus, the medical personnel cannot successfully insert the endotracheal tube through the endotracheal tube passage and into the trachea, or the soft tissue in the throat may be punctured by the tip of the endotracheal tube when the tube is inserted. As the current laryngoscope does not allow control of the direction of the tip of the endotracheal tube, the endotracheal tube will be inserted arbitrarily into the patient's upper respiratory tract during endotracheal tube intubation. Such an insertion may cause injury to the patient's upper respiratory tract during the endotracheal tube intubation. In the medical literature, the problem of the endotracheal tube puncturing the patient's respiratory tract is addressed, so the tracheal insertion tool of the prior art must be improved.

Furthermore, as disclosed in Taiwan patent TW M315097, although the endotracheal tube placement system in the prior art has two cameras to capture two different images, TW M315097 only discloses that the user first observes an image captured by a first image capturing unit and then inserts an endotracheal tube and a second image capturing unit covered therein. When the endotracheal tube blocks the field of vision of the first image capturing unit, the operator can observe a second image captured by the second image capturing unit to assist in locating the trachea. After the location of the trachea is correctly determined, the endotracheal tube is inserted to complete the intubation. But the dual-view endotracheal tube placement system of the prior art does not allow control of the direction of the endotracheal tube because the image of the upper respiratory tract of the human body must be seen. Therefore, in the process of inserting the endotracheal tube, the tip of the endotracheal tube may injure the soft tissue in the throat. It cannot solve the existing problems of the prior art.

### SUMMARY OF THE INVENTION

It is an object of the present invention to provide an image captured by a camera to position the tip of the endotracheal tube moving along the guiding portion continuously when the endotracheal tube is moving.

To achieve the abovementioned object, an endotracheal intubation system of the present invention is used for inserting an endotracheal tube into a human body, wherein the endotracheal tube comprises a tip. The endotracheal intubation system comprises a first camera, a blade, a second camera, and a display. The blade comprises a camera accommodation, an endotracheal tube passage, and a guiding portion. The first camera is situated in the camera accommodation for capturing a first image. The endotracheal tube passage comprises a side surface and a top surface, and the guiding portion is a junction of the side surface and the top surface. The second camera is situated in the endotracheal tube, wherein when the endotracheal tube moves in the endotracheal tube passage, the second camera captures an image of the guiding portion. The display is electrically connected to the first camera and the second camera for displaying the first image and the image of the guiding portion so as to ensure that the tip of the endotracheal tube moves along the guiding portion when the endotracheal tube moves along the endotracheal tube passage.

With the design of capturing the image of the guiding portion by the camera to control the direction of the front end of the endotracheal tube, the problem of the front end of the endotracheal tube puncturing the patient when the endotracheal tube is moving within the patient's trachea can be avoided, and the accuracy of tracheal intubation can also be improved.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1A illustrates a schematic diagram of a first embodiment of the endotracheal intubation system of the present invention.

FIG. 1B illustrates a schematic diagram of a second embodiment of the endotracheal intubation system of the present invention.

FIG. 1C illustrates a schematic diagram of a third embodiment of the endotracheal intubation system of the present invention.

FIG. 2 illustrates a schematic diagram of the blade of the first embodiment of the present invention.

FIG. 3 illustrates a usage stage diagram of the blade of the first embodiment of the present invention.

FIG. 4 illustrates a schematic diagram of the blade of the second embodiment of the present invention.

FIG. 5 illustrates a usage stage diagram of the blade of the second embodiment of the present invention.

### DETAILED DESCRIPTION OF THE PREFERRED EMBODIMENT

These and other objects and advantages of the present invention will become apparent from the following descriptions of the accompanying drawings, which disclose several embodiments of the present invention. It is to be understood that the drawings are to be used for purposes of illustration only, and not as a definition of the invention.

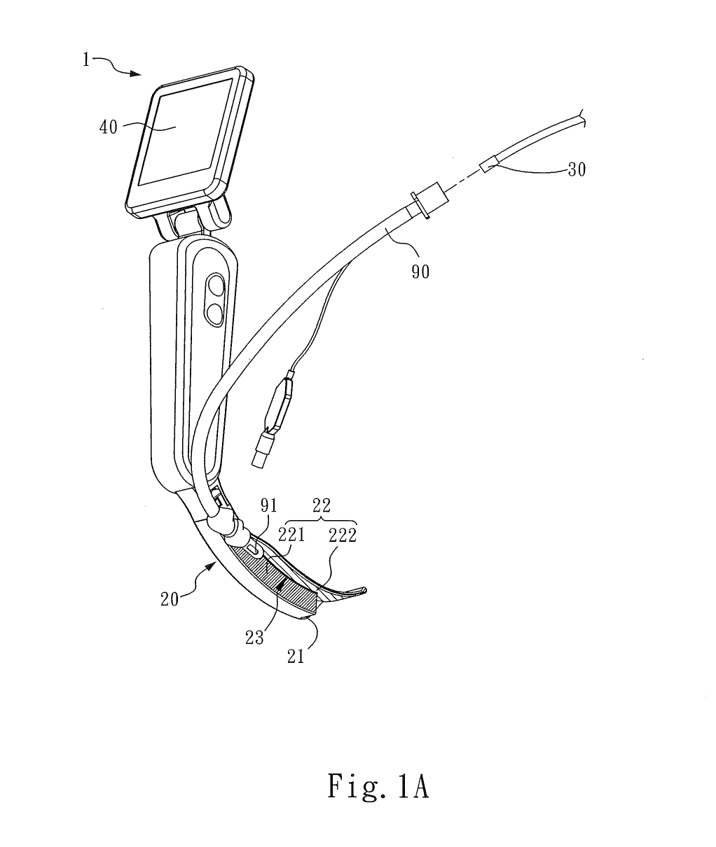

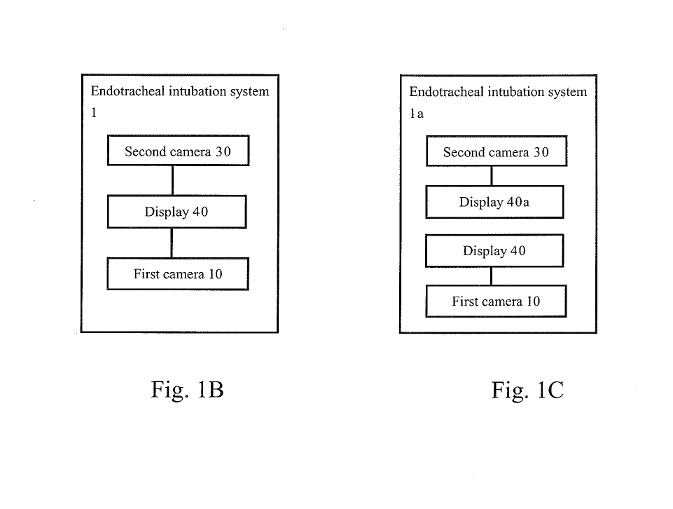

Please refer to FIG. 1A, 2B, 1C, and 3 to 5 respectively for schematic diagrams of the first, second, and third embodiments of the endotracheal intubation system; a schematic diagram of the blade of the first embodiment; a usage stage diagram of the blade of the first embodiment; a schematic diagram of the blade of the second embodiment; and a usage stage diagram of the blade of the second embodiment of the present invention.

As shown in FIG. 1A and 1B, the endotracheal intubation system 1 of the present invention is used for inserting an endotracheal tube 90 into an upper respiratory tract of a human body. The endotracheal tube 90 comprises a tip 91. As shown in FIG. 1A, in the first embodiment, the endotracheal intubation system 1 comprises a first camera 10, a blade 20, a second camera 30 and a display 40. The blade 20 comprises a camera accommodation 21, an endotracheal tube passage 22 and a guiding portion 23, wherein the first camera 10 is disposed at the camera accommodation 21 for capturing a first image to display on the display 40, wherein the first image is an image of the upper respiratory tract of a patient.

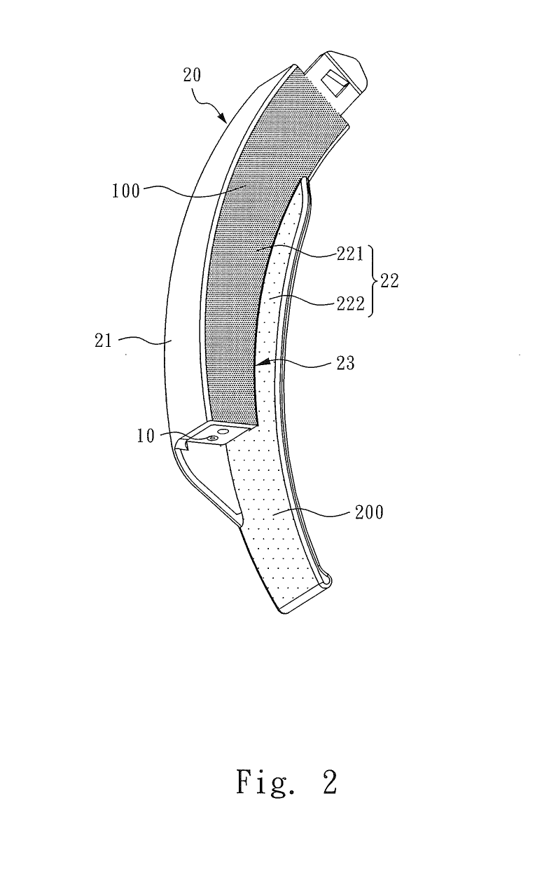

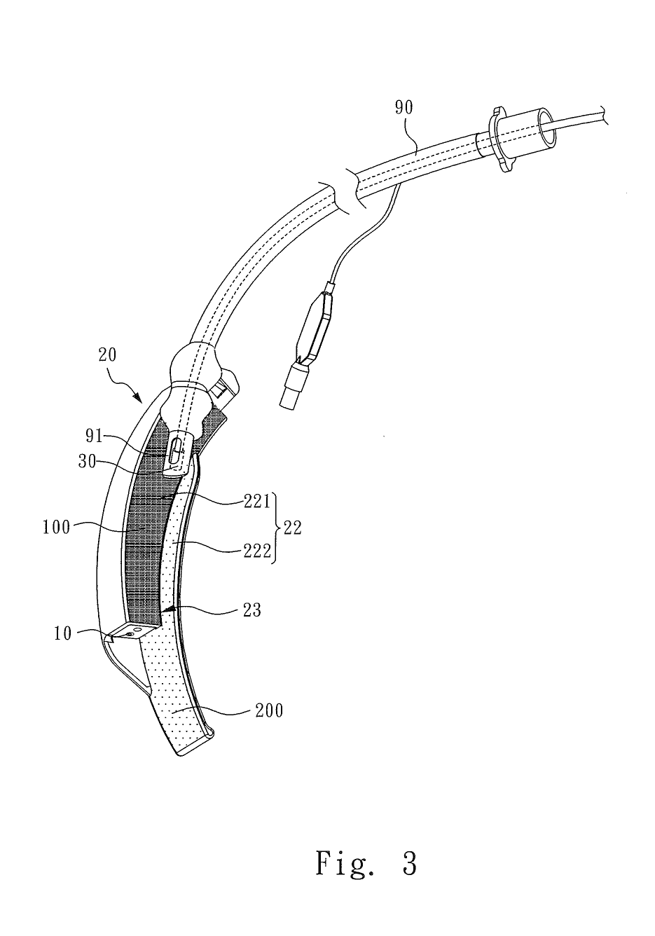

As shown in FIG. 2 and FIG. 3, the endotracheal tube passage 22 comprises a side surface 221 and a top surface 222. The guiding portion 23 is situated at a junction of the side surface 221 and the top surface 222. In the present embodiment, the side surface 221 has a first color 100 and the top surface 222 has a second color 200, wherein the first color 100 is visibly different from the second color 200. The second camera 30 is situated in the endotracheal tube 90. When the endotracheal tube 90 is moving within the endotracheal tube passage 20, the second camera 30 is used for capturing an image of the guiding portion 23 to display on the display 40. In the present embodiment, the image of the guiding portion 23 displayed by the display 40 is the image of the junction of the first color 100 and the second color 200. Therefore, when a medical worker operates the endotracheal intubation system 1 of the present invention to capture the image of the patient's trachea with the first camera 10, it can be ensured that the tip 91 is continuously moving along the guiding portion 23 as long as the medical worker can see the image of the first color 100, the second color 200 and the junction (the guiding portion 23) on the display 40 continuously, so as to achieve the effect of positioning the tip 91. As a result, the problem of the front end of the endotracheal tube 90 puncturing the patient in the tracheal intubation process mentioned in the prior art can be avoided, and also the accuracy of tracheal intubation can be improved. It is noted that the first color 100 and the second color 200 are not particularly limited; the two colors need only be visibly different such that the medical worker can clearly see the junction of the first color 100 and the second color 200 to achieve the purpose of guiding the tip 91 continuously along the guiding portion 23.

It is noted that in the present embodiment as shown in FIG. 1A and 1B, only one display 40 is electrically connected to the first camera 10 and the second camera 30 to display the first image and the image of the guiding portion 23. That is, the screen of the display 40 is divided into two sub-screens, of which one sub-screen shows the first image (the image of the upper respiratory tract of the patient) and the other sub-screen shows the image of the guiding portion 23, but the present invention is not limited thereto. As shown in FIG. 1C, according to another embodiment of the present invention, there are two displays, i.e., display 40 and display 40a, wherein the first image and the image of the guiding portion 23 are shown on the displays 40, 40a respectively. In other words, in the present embodiment, the endotracheal intubation system 1a has two displays, the first image and the image of the guiding portion 23 are displayed on different displays, and the displays 40, 40a each display only a single picture.

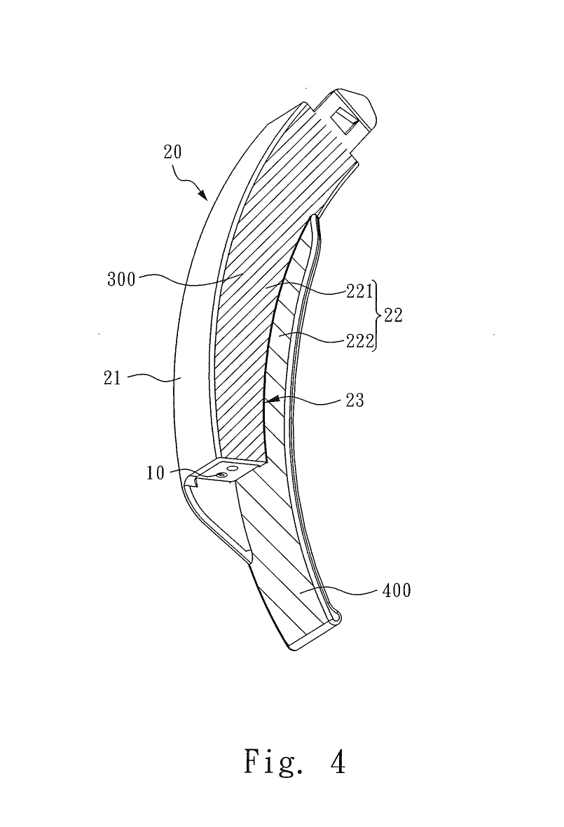

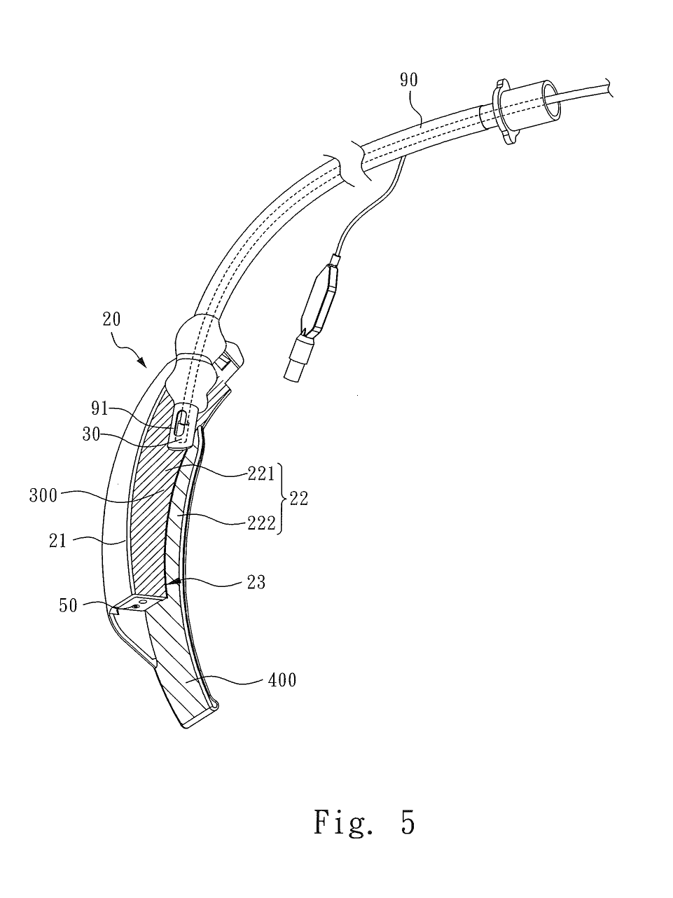

According to another embodiment of the present invention, as shown in FIG. 4, the side surface 221 has a first pattern 300 and the top surface 222 has a second pattern 400, wherein the first pattern 300 is different from the second pattern 400. The first pattern 300 and the second pattern 400 can be different geometric patterns, or wavy lines and the like. As shown in FIG. 5, when the first camera 10 captures the patient's trachea and the endotracheal tube 90 is moving within the endotracheal tube passage 20, the image of the guiding portion 23 shown on the display 40 is a junction (the guiding portion 23) between the first pattern 300 and the second pattern 400 so as to ensure that the tip 91 moves along the guiding portion 23 to achieve the effect of positioning the tip 91. It is noted that the first pattern 300 and the second pattern 400 are not particularly limited; the two patterns need only be sufficiently different that the medical personnel can clearly see the junction of the first pattern 300 and the second pattern 400 so as to ensure that the tip 91 moves continuously along the guiding portion 23 and to achieve the purpose of positioning the tip 91. It should be noted that the variation of the guiding portion 23 is not limited to the aforementioned embodiments. The guiding portion 23 can also be a colored line and is not limited to a line demarking different colors or to the intersection of different patterns.

The endotracheal intubation system 1 of the present invention is designed to capture an image of the patient's trachea with the first camera 10 and capture an image of the guiding portion 32 with the second camera 30 so that the front end of the endotracheal tube 90 can be positioned by using the image of the guiding portion 23 captured by the second camera 30. Thus, the present invention can prevent the problem of front end of the endotracheal tube 90 puncturing the patient when the endotracheal tube is moving within the patient's trachea, and the accuracy of tracheal intubation can also be improved.

It is noted that the above-mentioned embodiments are only for illustration. It is intended that the present invention cover modifications and variations of this invention provided they fall within the scope of the following claims and their equivalents. Therefore, it will be apparent to those skilled in the art that various modifications and variations can be made to the structure of the present invention without departing from the scope of the invention.

What is claimed is:

1. An endotracheal intubation system used for inserting an endotracheal tube into an upper respiratory tract of a human body, wherein the endotracheal tube comprises a tip, the endotracheal intubation system comprising:
a first camera, for capturing a first image;
a blade, comprising a camera accommodation, an endotracheal tube passage, and a guiding portion, wherein the first camera is situated in the camera accommodation, and wherein the endotracheal tube passage comprises a side surface and a top surface, the guiding portion being a junction of the side surface and the top surface;
a second camera, situated in the endotracheal tube, wherein when the endotracheal tube is moving in the endotracheal tube passage, the second camera is used for capturing an image of the guiding portion, and
at least one display, electrically connected to the first camera and the second camera for displaying the first image and the image of the guiding portion so as to ensure that the tip of the endotracheal tube moves along the guiding portion when the endotracheal tube moves along the endotracheal tube passage.

2. The endotracheal intubation system as claimed in claim 1, wherein the at least one display are two displays for displaying the images of the guiding portion and the first image separately.

3. The endotracheal intubation system as claimed in claim 2, wherein the first image is an image of the upper respiratory tract of a human body.

4. The endotracheal intubation system as claimed in claim 1, the side surface having a first color and the top surface having a second color, wherein the first color is different from the second color.

5. The endotracheal intubation system as claimed in claim 4, wherein the at least one display are two displays for displaying the images of the guiding portion and the first image separately.

6. The endotracheal intubation system as claimed in claim 5, wherein the first image is an image of the upper respiratory tract of a human body.

7. The endotracheal intubation system as claimed in claim 4, wherein when the endotracheal tube is moving along the endotracheal tube passage, the image guiding portion displayed by the at least one display is a junction of the first color and the second color.

8. The endotracheal intubation system as claimed in claim 7, wherein the at least one display are two displays for displaying the images of the guiding portion and the first image separately.

9. The endotracheal intubation system as claimed in claim 8, wherein the first image is an image of the upper respiratory tract of a human body.

10. The endotracheal intubation system as claimed in claim 1, the side surface having a first pattern and the top surface having a second pattern, wherein the first pattern is different from the second pattern.

11. The endotracheal intubation system as claimed in claim 10, wherein the at least one display are two displays for displaying the images of the guiding portion and the first image separately.

12. The endotracheal intubation system as claimed in claim 11, wherein the first image is an image of the upper respiratory tract of a human body.

13. The endotracheal intubation system as claimed in claim 10, wherein when the endotracheal tube is moving along the endotracheal tube passage, the image guiding portion displayed by the at least one display is a junction of the first pattern and the second pattern.

14. The endotracheal intubation system as claimed in claim 13, wherein the at least one display are two displays for displaying the images of the guiding portion and the first image separately.

15. The endotracheal intubation system as claimed in claim 14, wherein the first image is an image of the upper respiratory tract of a human body.
